(19) United States
(12) Patent Application Publication
(10) Pub. No.: US 2022/0409764 A1
(43) Pub. Date: Dec. 29, 2022

(54) DISINFECTING DEVICE FOR SURGICAL SMOKE EVACUATION SYSTEM

(71) Applicant: Covidien LP, Mansfield, MA (US)

(72) Inventors: K V S Manoj Kumar Vadali, Hyderabad (IN); Sarita Rai, Pune (IN)

(21) Appl. No.: 17/359,976

(22) Filed: Jun. 28, 2021

(51) Int. Cl. A61L 9/20; A61B 18/00

(52) U.S. Cl. CPC A61L 9/20; A61B 18/00; A61L 2209/12; A61L 2209/111; A61B 2218/008

(57) ABSTRACT

A surgical smoke evacuation system includes a smoke evacuator, a suction instrument, a suction conduit, and a disinfecting device. The smoke evacuator includes a suction generator configured to create a vacuum force. The suction instrument is configured to couple to the smoke evacuator via the suction conduit. The disinfecting device is operably coupled to the suction conduit or the smoke evacuator. The disinfecting device includes a housing defining an inner chamber, an inlet extending from a distal end of the housing and in fluid communication with the inner chamber, an outlet extending from a proximal end of the housing and in fluid communication with the inner chamber, and an ultraviolet light emitting diode board. The ultraviolet light emitting diode board includes at least one ultraviolet-c light emitting diode disposed in the inner chamber and configured to disinfect surgical smoke within the inner chamber.

### FIELD

The present disclosure generally relates to ultraviolet-c radiation disinfecting devices for use with surgical smoke evacuation systems, and surgical smoke evacuation systems including ultraviolet-c radiation disinfecting devices.

### BACKGROUND

Minimally invasive surgical procedures including both endoscopic and laparoscopic procedures permit surgery to be performed on organs, tissues and vessels far removed from an opening within the tissue. In laparoscopic procedures, the abdominal cavity is insufflated with an insufflation gas, e.g., CO₂, to create a pneumoperitoneum thereby providing access to the underlying organs. A laparoscopic instrument is introduced through a cannula accessing the abdominal cavity to perform one or more surgical tasks. The cannula may incorporate a seal to establish a substantially fluid tight seal about the instrument to preserve the integrity of the pneumoperitoneum.

Instruments utilized during a laparoscopic procedure may include lasers, electro-cautery or sonic cutting instruments, which produce smoke and/or an aerosol as a byproduct of treating tissue. Smoke plumes can obscure the clinician's field of vision and the odor generated is unpleasant. Further, the smoke plume may contain infectious agents which may contaminate the operating arena thereby presenting a danger to operating personnel. The chemical vapor may be irritating to the respiratory tract and also may be carcinogenic. The smoke, noxious fumes, and other gases and vapors can include particulates, bacteria, viral elements, and undesirable odors.

Conventional methodologies for evacuating smoke include using a surgical smoke evacuation device. This device includes a vacuum pump, tubing, and a filter to filter out particulates and microbials and properly dispose of them. A tube is typically attached to an insufflation port of an access cannula or to evacuation tubing of a surgical instrument and the smoke is ventilated through the filter. These filters are limited in their filtration capabilities, are prone to clogging, and must be replaced regularly.

### SUMMARY

The present disclosure relates to ultraviolet-c radiation disinfecting devices for use with surgical smoke evacuation systems, and surgical smoke evacuation systems including ultraviolet-c radiation disinfecting devices.

In an aspect of the disclosure, a surgical smoke evacuation system is disclosed. The surgical smoke evacuation system includes a smoke evacuator, a suction instrument, a suction conduit, and a disinfecting device. The smoke evacuator includes a suction generator configured to create a vacuum force. The suction instrument is configured to couple to the smoke evacuator via the suction conduit. The disinfecting device is operably coupled to at least one of the suction conduit or the smoke evacuator. The disinfecting device includes a housing defining an inner chamber, an inlet extending from a distal end of the housing and in fluid communication with the inner chamber, an outlet extending from a proximal end of the housing and in fluid communication with the inner chamber, and an ultraviolet light emitting diode board. The ultraviolet light emitting diode board includes at least one ultraviolet-c light emitting diode disposed in the inner chamber of the housing and configured to disinfect surgical smoke within the inner chamber of the housing.

In an aspect, the disinfecting device is disposed within the smoke evacuator.

In an aspect, the inlet of the housing includes a barbed outer surface configured to engage with at least one of the suction instrument or the suction conduit and the outlet of the housing includes a barbed outer surface configured to engage with at least one of an inlet port of the smoke evacuator or the suction conduit.

In an aspect, the inlet of the housing includes a smooth outer surface configured to friction-fit to at least one of the suction instrument or the suction conduit and the outlet of the housing includes a smooth outer surface configured to friction-fit to at least one of an inlet port of the smoke evacuator or the suction conduit.

In an aspect, the disinfecting device includes a power cable extending from the housing and configured to electrically couple the ultraviolet light emitting diode board to a power source.

In an aspect, at least a portion of the housing includes conductive terminals extending externally therefrom and configured to electrically couple a positive terminal and a negative terminal of the ultraviolet light emitting diode board to a power source.

In an aspect, the suction conduit includes a proximal side and a distal side. The proximal side includes a first outlet port configured to couple to a first inlet port of the smoke evacuator, a second outlet port configured to couple to a second inlet port of the smoke evacuator, and a third outlet port configured to couple to a third inlet port of the smoke evacuator. The distal side includes a first inlet port configured to couple to the suction instrument, a second inlet port configured to couple to a second suction instrument, and a third inlet port configured to couple to a third suction instrument. Additionally, or alternatively, the disinfecting device may be disposed between the proximal side and the distal side of the suction conduit such that smoke passing through each of the first inlet port, the second inlet port, and the third inlet port of the distal side passes through the disinfecting device. Additionally, or alternatively, the surgical smoke evacuation system may include a cap configured to couple to at least one of the first inlet port, the second inlet port, or the third inlet port of the distal side of the suction conduit.

In an aspect, the suction generator includes at least one ultraviolet-c light emitting diode configured to disinfect surgical smoke vacuumed through the suction generator.

In another aspect of the present disclosure, a disinfecting device is disclosed. The disinfecting device includes a housing defining an inner chamber, an inlet, an outlet, and an ultraviolet light emitting diode board. The inlet extends from a distal end of the housing and is in fluid communication with the inner chamber. The outlet extends from a proximal end of the housing and is in fluid communication with the inner chamber. The ultraviolet light emitting diode board includes at least one ultraviolet-c light emitting diode disposed in the inner chamber of the housing. The at least one ultraviolet-c light emitting diode is configured to disinfect surgical smoke within the inner chamber of the housing.

In an aspect, the inlet of the housing includes a barbed outer surface configured to engage with at least one of a suction instrument or a suction conduit and the outlet of the housing includes a barbed outer surface configured to engage with at least one of an inlet port of a smoke evacuator or a suction conduit.

In an aspect, the inlet of the housing includes a smooth outer surface configured to friction-fit to at least one of a suction instrument or a suction conduit and the outlet of the housing includes a smooth outer surface configured to friction-fit to at least one of an inlet port of a smoke evacuator or a suction conduit.

In an aspect, the disinfecting device further includes a power cable extending from the housing and configured to electrically couple the ultraviolet light emitting diode board to a power source.

In an aspect, the disinfecting device further includes a battery operably coupled to the ultraviolet light emitting diode board and at least one of an automatic sensor or a manual switch configured to activate the at least one ultraviolet-c light emitting diode of the ultraviolet light emitting diode board.

In an aspect, at least a portion of the housing includes conductive terminals extending externally therefrom and configured to electrically couple a positive terminal and a negative terminal of the ultraviolet light emitting diode board to a power source.

In another aspect of the present disclosure, a suction conduit is provided. The suction conduit includes a proximal side including a first inlet port, a second inlet port, and a third inlet port, a distal side including a first outlet port, a second outlet port, and a third outlet port, and a disinfecting device disposed between the proximal side and the distal side. The first outlet port is configured to couple to a first inlet port of a smoke evacuator, the second outlet port is configured to couple to a second inlet port of the smoke evacuator, and the third outlet port is configured to couple to a third inlet port of the smoke evacuator. The first inlet port is configured to couple to a first suction instrument, the second inlet port is configured to couple to a second suction instrument, and the third inlet port is configured to couple to a third suction instrument. The disinfecting device is disposed between the proximal side and the distal side of the suction conduit such that smoke passing through each of the first inlet port, the second inlet port, and the third inlet port of the distal side passes through the disinfecting device.

In an aspect, the disinfecting device includes an ultraviolet light emitting diode board including at least one ultraviolet-c light emitting diode configured to disinfect surgical smoke.

In an aspect, the disinfecting device includes a housing defining an inner chamber, an inlet extending from a distal end of the housing and in fluid communication with the inner chamber, and an outlet extending from a proximal end of the housing and in fluid communication with the inner chamber. The at least one ultraviolet-c light emitting diode is disposed within the inner chamber.

In an aspect, at least a portion of the housing includes conductive terminals extending externally therefrom and configured to electrically couple a positive terminal and a negative terminal of the ultraviolet light emitting diode board to a power source.

In an aspect, the suction conduit includes a cap configured to couple to at least one of the first inlet port, the second inlet port, or the third inlet port of the distal side of the suction conduit.

Other features of the disclosure will be appreciated from the following description.

### DESCRIPTION OF THE DRAWINGS

The accompanying drawings, which are incorporated in and constitute a part of this specification, illustrate aspects and features of the disclosure and, together with the detailed description below, serve to further explain the disclosure, in which:

FIG. 1 is a perspective view of a surgical smoke evacuation system in accordance with an aspect of the present disclosure;

FIG. 2 is side, cross-sectional, view of a disinfecting device in accordance with an aspect of the present disclosure;

FIG. 3 is a front perspective view of a smoke evacuator and a multi-port suction conduit including a disinfecting device in accordance with an aspect of the present disclosure;

FIG. 4 is a side view of the multi-port suction conduit and disinfecting device of FIG. 3; and

FIG. 5 is a front view of a suction generator of a smoke evacuator including a disinfecting device in accordance with an aspect of the present disclosure.

### DETAILED DESCRIPTION

Aspects of the disclosure are described hereinbelow with reference to the accompanying drawings; however, it is to be understood that the disclosed aspects are merely exemplary of the disclosure and may be embodied in various forms. Well-known functions or constructions are not described in detail to avoid obscuring the disclosure in unnecessary detail. Therefore, specific structural and functional details disclosed herein are not to be interpreted as limiting, but merely as a basis for the claims and as a representative basis for teaching one skilled in the art to variously employ the disclosure in virtually any appropriately detailed structure.

Descriptions of technical features of an aspect of the disclosure should typically be considered as available and applicable to other similar features of another aspect of the disclosure. Accordingly, technical features described herein according to one aspect of the disclosure may be applicable to other aspects of the disclosure, and thus duplicative descriptions may be omitted herein. Like reference numerals may refer to like elements throughout the specification and drawings.

In order to reduce the risk of virus spread with the removal of insufflation gas during laparoscopy, the use of devices to filter the released insufflation gas is recommended. The SARS-Cov-2 RNA virus has been reported to have a size of 0.06-0.14 microns, with HIV having a size of 0.12 microns, HPV having a size of 0.055 microns, and hepatitis C having a size of 0.06 microns. Smoke evacuator products typically utilize Ultra Low Particulate Air (ULPA) filters to filter the surgical smoke and other removed insufflation gases. A major challenge with ULPA filters is that the effectiveness to filter viruses such as the COVID virus is not certain. Additionally, the filters must be regularly replaced in the product to continue performing the surgical procedure and to continue using the surgical smoke evacuation system.

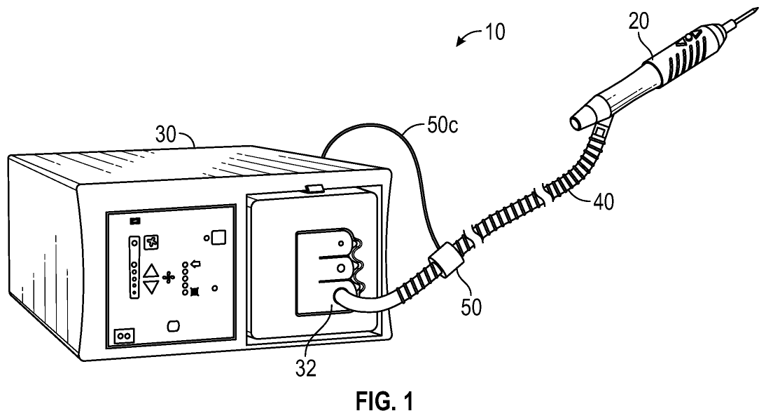

FIG. 1 illustrates a surgical smoke evacuation system 10 including a suction instrument 20, a smoke evacuator 30, a suction conduit 40 connecting the suction instrument 20 to the smoke evacuator 30, and a disinfecting device 50. The suction instrument 20 may be any surgical instrument that includes a suction port for removing surgical smoke and other debris from a surgical site, which may or may not include a surgical treatment member such as an electrode for treating tissue. The smoke evacuator 30 includes a suction generator (e.g., suction generator 35 in FIG. 5) that creates a vacuum force. The suction conduit 40 is connected to the suction instrument 20 at one end and to an inlet port 32 of the smoke evacuator 30 at its other end.

The disinfecting device 50 may be a separate module that is removably or permanently connected to any of the suction instrument 20, the suction conduit 40, or the inlet port 32 of the smoke evacuator 30, and/or may be incorporated in the smoke evacuator 30. For example, and for illustrative purposes, FIG. 1 illustrates the disinfecting device 50 as a module connected to the suction conduit 40, between the suction instrument 20 and the inlet port 32 of the smoke evacuator 30. In addition, or alternatively, a component of the smoke evacuator 30 may include one or more disinfecting devices, similar to disinfecting device 50, along its fluid vacuum path. For example, FIG. 5 illustrates an example suction generator 35, which is the component of the smoke evacuator 30 that creates the negative pressure enabling smoke removal from a surgical site, that includes at least one disinfecting ultraviolet-c radiating light emitting diode 39 for disinfecting surgical smoke entering or departing the smoke evacuator 30. The disinfecting device 50 may be used in conjunction with suction generator 35 or suction generator 35 may be used without a separate disinfecting device 50.

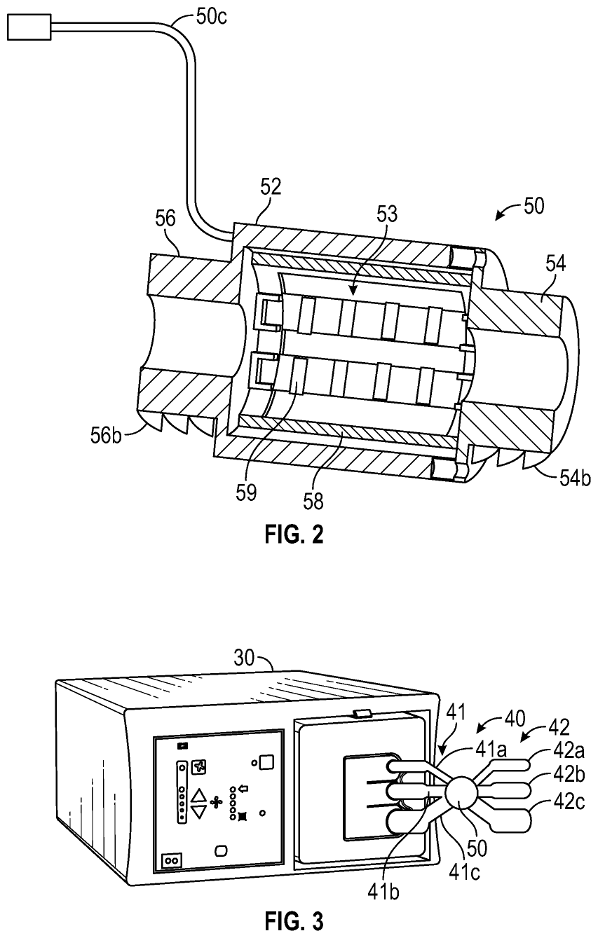

With continued reference to FIG. 1, and with additional reference to FIG. 2, the disinfecting device 50 includes a housing 52 defining an inner chamber 53, an inlet 54 extending from a distal end 52b of the housing 52, and an outlet 56 extending from a proximal end 52a of the housing 52. Although illustrated and described as extending from the proximal end 52a and distal end 52b of the housing 52, inlet 54 and outlet 56 may be disposed anywhere along the length of housing 52 and may extend radially outward from its longitudinal axis. The inlet 54 of the housing 52 couples to the suction instrument 20, either directly or via the suction conduit 40. The outlet 56 couples to the inlet port 32 of the smoke evacuator 30, either directly or via the suction conduit 40. The inlet 54 and the outlet 56 of the housing 52 can couple to the suction conduit 40, the inlet port 32 of the smoke evacuator 30, or the suction instrument 20 in various different manners. For example, in an aspect, the inlet 54 of the housing 52 includes a barbed outer surface 54b configured to engage with at least one of the suction instrument 20 or the suction conduit 40. Additionally, or alternatively, the outlet 56 of the housing 52 may include a barbed outer surface 56b configured to engage with at least one of an inlet port 32 of the smoke evacuator 30 or the suction conduit 40. In another aspect, the inlet 54 of the housing 52 includes a smooth outer surface (not shown) configured to friction-fit to at least one of the suction instrument 20 or the suction conduit 40. Additionally, or alternatively, the outlet 56 of the housing 52 may include a smooth outer surface (not shown) configured to friction-fit to at least one of an inlet port 32 of the smoke evacuator 30 or the suction conduit 40.

Each of the inlet 54 and the outlet 56 are in fluid communication with the inner chamber 53 of the housing 52 such that gas or liquid vacuumed into the inlet 54 from the suction instrument 20 passes through the inner chamber 53 before exiting through the outlet 56 to the smoke evacuator 30. Additionally, the disinfecting device 50 includes an ultraviolet light emitting diode board 58 disposed within the inner chamber 53 of the housing 52 which serves to disinfect the surgical smoke present in the inner chamber 53 as it passes through the inner chamber 53 from the inlet 54 to the outlet 56. The ultraviolet light emitting diode board 58 includes one or more ultraviolet-c light emitting diodes 59. The ultraviolet-c light emitting diodes 59 emit ultraviolet-c radiation (e.g., at a wavelength of approximately 254 nm) which is effective in killing a range of bacteria and viruses, such as the COVID-19 causing virus, within a short time by breaking the virus's DNA.

The ultraviolet-c light emitting diodes 59 of the ultraviolet light emitting diode board 58 may be powered by a battery included in the disinfecting device 50 itself or by an external power source. For example, in an aspect, the disinfecting device includes terminal connections or a cable 50c (FIG. 1) that can be connected to an external power source, such as a communication port or USB input of the smoke evacuator 30. Positive and negative terminal connections that are electrically coupled to the ultraviolet light emitting diode board 58 may extend through one or both of the inlet 54 or the outlet 56 of the housing 52, or any other portion of the housing 52, to electrically connect the ultraviolet light emitting diode board 58 to a power source.

FIG. 3 illustrates a smoke evacuator 30 that includes multiple inlet ports 32 for connecting to multiple suction instruments 20. Each inlet port of the multiple inlet ports 32 may have a different diameter for connecting to different sized suction conduits 40. In particular, the smoke evacuator 30 in FIG. 3 includes a first inlet port 31a, a second inlet port 31b, and a third inlet port 31c. Although illustrated and described as having three inlet ports 32, smoke evacuator 30 may have any number of inlet ports 32 for connecting to any number of suction instruments 20. For smoke evacuators 30 that include more than one inlet port 32, a separate disinfecting device 50 may be coupled to, or in fluid communication with, each inlet port of the plurality of inlet ports 32. For example, a first disinfecting device 50 may be coupled to the first inlet port 31a, a second disinfecting device 50 may be coupled to the second inlet port 31b, and a third disinfecting device 50 may be coupled to the third inlet port 31c. Alternatively, to reduce the number of disinfecting devices 50 used, a multi-port suction conduit 45 may be utilized to connect each of first inlet port 31a, second inlet port 31b, and third inlet port 31c to three different suction instruments 20 that pass the surgical smoke through a single disinfecting device 50.

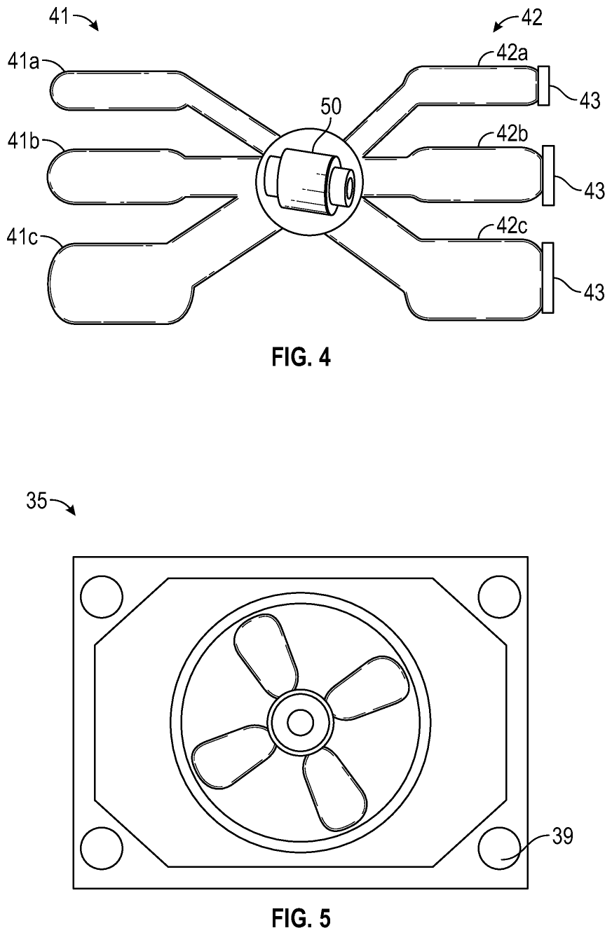

In particular, FIG. 4 illustrates a multi-port suction conduit 45 including a proximal side 41 and a distal side 42, with a disinfecting device 50 positioned between the proximal side 41 and the distal side 42. The proximal side 41 includes a first outlet port 41a configured to couple to a first inlet port 31a of a smoke evacuator 30, a second outlet port 41b configured to couple to a second inlet port 31b of the smoke evacuator 30, and a third outlet port 41c configured to couple to a third inlet port 31c of the smoke evacuator 30. The distal side 42 includes a first inlet port 42a configured to couple to a first suction instrument (e.g., suction instrument 20), a second inlet port 42b configured to couple to a second suction instrument (not shown), and a third inlet port 42c configured to couple to a third suction instrument (not shown). The disinfecting device 50 is disposed between the proximal side 41 and the distal side 42 of the multi-port suction conduit 45 such that smoke passing through each of the first inlet port 42a, the second inlet port 42b, and the third inlet port 42c of the distal side 42 passes through the disinfecting device 50 before being vacuumed to the proximal side 41 (e.g., via the first outlet port 41a, second outlet port 41b, and third outlet port 41c). One or more caps 43 can be used to plug any of first inlet port 42a, second inlet port 42b, or third inlet port 42c of multi-port suction conduit 45 or first inlet 31a, second inlet 31b, or third inlet 31c of smoke evacuator 30 when the inlet or inlet port is not in use.

Persons skilled in the art will understand that the devices and methods specifically described herein and illustrated in the accompanying drawings are non-limiting. It is envisioned that the elements and features may be combined with the elements and features of another without departing from the scope of the disclosure. As well, one skilled in the art will appreciate further features and advantages of the disclosure.

What is claimed is:

1. A surgical smoke evacuation system, comprising:
a smoke evacuator including a suction generator configured to create a vacuum force;
a suction instrument configured to couple to the smoke evacuator;
a suction conduit configured to couple the suction instrument to the smoke evacuator; and
a disinfecting device operably coupled to at least one of the suction conduit or the smoke evacuator, the disinfecting device including:
a housing defining an inner chamber;
an inlet extending from a distal end of the housing and in fluid communication with the inner chamber;
an outlet extending from a proximal end of the housing and in fluid communication with the inner chamber; and
an ultraviolet light emitting diode board including at least one ultraviolet-c light emitting diode disposed in the inner chamber of the housing, the at least one ultraviolet-c light emitting diode configured to disinfect surgical smoke within the inner chamber of the housing.

2. The smoke evacuation system according to claim 1, wherein the disinfecting device is disposed within the smoke evacuator.

3. The smoke evacuation system according to claim 1, wherein:
the inlet of the housing includes a barbed outer surface configured to engage with at least one of the suction instrument or the suction conduit; and
the outlet of the housing includes a barbed outer surface configured to engage with at least one of an inlet port of the smoke evacuator or the suction conduit.

4. The smoke evacuation system according to claim 1, wherein:
the inlet of the housing includes a smooth outer surface configured to friction-fit to at least one of the suction instrument or the suction conduit; and
the outlet of the housing includes a smooth outer surface configured to friction-fit to at least one of an inlet port of the smoke evacuator or the suction conduit.

5. The smoke evacuation system according to claim 1, wherein the disinfecting device includes a power cable extending from the housing and configured to electrically couple the ultraviolet light emitting diode board to a power source.

6. The smoke evacuation system according to claim 1, wherein at least a portion of the housing includes conductive terminals extending externally therefrom and configured to electrically couple a positive terminal and a negative terminal of the ultraviolet light emitting diode board to a power source.

7. The smoke evacuation system according to claim 1, wherein the suction conduit includes:
a proximal side including a first outlet port configured to couple to a first inlet port of the smoke evacuator, a second outlet port configured to couple to a second inlet port of the smoke evacuator, and a third outlet port configured to couple to a third inlet port of the smoke evacuator; and
a distal side including a first inlet port configured to couple to the suction instrument, a second inlet port configured to couple to a second suction instrument, and a third inlet port configured to couple to a third suction instrument, wherein the disinfecting device is disposed between the proximal side and the distal side of the suction conduit such that smoke passing through each of the first inlet port, the second inlet port, and the third inlet port of the distal side passes through the disinfecting device.

8. The smoke evacuation system according to claim 7, further comprising a cap configured to couple to at least one of the first inlet port, the second inlet port, or the third inlet port of the distal side of the suction conduit.

9. The smoke evacuation system according to claim 1, wherein the suction generator includes at least one ultraviolet-c light emitting diode configured to disinfect surgical smoke vacuumed through the suction generator.

10. A disinfecting device for a smoke evacuation system, the disinfecting device comprising:
a housing defining an inner chamber;
an inlet extending from a distal end of the housing and in fluid communication with the inner chamber;
an outlet extending from a proximal end of the housing and in fluid communication with the inner chamber; and
an ultraviolet light emitting diode board including at least one ultraviolet-c light emitting diode disposed in the inner chamber of the housing, the at least one ultraviolet-c light emitting diode configured to disinfect surgical smoke within the inner chamber of the housing.

11. The disinfecting device according to claim 10, wherein:
the inlet of the housing includes a barbed outer surface configured to engage with at least one of a suction instrument or a suction conduit; and
the outlet of the housing includes a barbed outer surface configured to engage with at least one of an inlet port of a smoke evacuator or a suction conduit.

12. The disinfecting device according to claim 10, wherein:
the inlet of the housing includes a smooth outer surface configured to friction-fit to at least one of a suction instrument or a suction conduit; and
the outlet of the housing includes a smooth outer surface configured to friction-fit to at least one of an inlet port of a smoke evacuator or a suction conduit.

13. The disinfecting device according to claim 10, further comprising a power cable extending from the housing and configured to electrically couple the ultraviolet light emitting diode board to a power source.

14. The disinfecting device according to claim 10, further comprising a battery operably coupled to the ultraviolet light emitting diode board and at least one of an automatic sensor or a manual switch configured to activate the at least one ultraviolet-c light emitting diode of the ultraviolet light emitting diode board.

15. The disinfecting device according to claim 10, wherein at least a portion of the housing includes conductive terminals extending externally therefrom and configured to electrically couple a positive terminal and a negative terminal of the ultraviolet light emitting diode board to a power source.

16. A suction conduit for a surgical smoke evacuation system, the suction conduit comprising:
a proximal side including:
a first outlet port configured to couple to a first inlet port of a smoke evacuator;
a second outlet port configured to couple to a second inlet port of the smoke evacuator; and
a third outlet port configured to couple to a third inlet port of the smoke evacuator;
a distal side including:
a first inlet port configured to couple to a first suction instrument;
a second inlet port configured to couple to a second suction instrument; and
a third inlet port configured to couple to a third suction instrument; and
a disinfecting device disposed between the proximal side and the distal side of the suction conduit such that smoke passing through each of the first inlet port, the second inlet port, and the third inlet port of the distal side passes through the disinfecting device.

17. The suction conduit according to claim 16, wherein the disinfecting device includes an ultraviolet light emitting diode board including at least one ultraviolet-c light emitting diode configured to disinfect surgical smoke.

18. The suction conduit according to claim 17, wherein the disinfecting device includes:
a housing defining an inner chamber;
an inlet extending from a distal end of the housing and in fluid communication with the inner chamber; and
an outlet extending from a proximal end of the housing and in fluid communication with the inner chamber, wherein the at least one ultraviolet-c light emitting diode is disposed within the inner chamber.

19. The suction conduit according to claim 18, wherein at least a portion of the housing includes conductive terminals extending externally therefrom and configured to electrically couple a positive terminal and a negative terminal of the ultraviolet light emitting diode board to a power source.

20. The suction conduit according to claim 16, further comprising a cap configured to couple to at least one of the first inlet port, the second inlet port, or the third inlet port of the distal side of the suction conduit.
